(19) United States
(12) Patent Application Publication
(10) Pub. No.: US 2020/0197276 A1
(43) Pub. Date: Jun. 25, 2020

(54) UV-Detectable Antibacterial Compositions

(71) Applicant: Three Kings Corporation, Corinth, MS (US)

(72) Inventor: Donald O King, Corinth, MS (US)

(73) Assignee: Three Kings Corporation, Corinth, MS (US)

(21) Appl. No.: 16/637,619

(22) PCT Filed: Aug. 9, 2018
(86) PCT No.: PCT/US18/46114

§ 371 Date: Feb. 7, 2020

Related U.S. Application Data
(60) Provisional application No. 62/605,356, filed on Aug. 9, 2017.

(51) Int. Cl. A61K 8/41; A61Q 17/00; A61Q 19/00; A61K 8/34

(52) U.S. Cl. CPC A61K 8/416; A61K 8/345; A61Q 19/00; A61Q 17/005

(57) ABSTRACT

An antimicrobial composition suitable for use on human skin and containing a UV-detectable compound that will allow for detection of the application of the composition, using a UV-light source. A kit comprising such an antimicrobial composition and a UV-light source.

### CROSS REFERENCE TO RELATED APPLICATIONS

U.S. Provisional Application 87/661,563.

### NAMES OF PARTIES TO A JOINT RESEARCH AGREEMENT

None.

### SEQUENCE LISTING

None.

### BACKGROUND OF THE INVENTION

### Field of the Invention

The present invention relates to an antibacterial composition for personal care that contains an additive that is detectable under UV light. More particularly, the composition may contain a quaternary ammonium compound, such as benzalkonium chloride or benzethonium chloride, as the active ingredient.

### Background of the Invention

Antibacterial personal care compositions are known in the art. Especially useful are antibacterial cleansing compositions, which typically used to clean the skin and destroy bacteria present on the skin, especially the hands of the user.

Antibacterial compositions for use on the skin are used, for example, in the food service industry, the health care industry, the food processing industry, and by individuals seeking to protect themselves from bacterial infections. The widespread use of antibacterial compositions indicates the importance consumers place on preventing disease caused by bacteria on their skin and the skin of their children.

In addition to an antibacterial active ingredient, antibacterial personal care compositions typically contain an anionic surfactant for cleansing and foam generation, skin conditioning agents, perfumes, and dyes. They may also contain thickening agents, such as clays, polymers, or colloids, any of which may enhance the feel of the composition for the user.

There are three basic types of antibacterial active ingredients for use on skin, namely, alcohols, quaternary ammonium compounds, and triclosan. Quaternary ammonium compounds have been used as the active ingredient or ingredients in many commercial antibacterial personal care compositions. They are primarily benzethonium chloride and benzalkonium chloride, each an FDA approved antibacterial compound. U.S. Pat. No. 5,994,383 describes a composition containing benzalkonium chloride and homologs thereof in compositions intended for treating a skin infection. U.S. Pat. No. 7,754,770 describes antibacterial compositions for personal care that contain benzalkonium chloride. Other patent documents describing hand sanitizers are U.S. Pat. No. 8,562,907, U.S. Patent Application 20170181429 A1, and EP Patent 2249773 B1, all of which are incorporated by reference.

However, antibacterial compositions can only be effective when they are used. They must be applied so as to cover the skin where the presence of bacteria and other microorganisms can exist and be transferred to the eyes, mouth, nose or other mucosal membranes, causing disease. Many users will effectively use such compositions in just such a way, but some, particularly children, will not. They may even avoid the use of antibacterial compositions such as hand sanitizers due to the feel or other sensory characteristics that are unpleasant to certain users.

Therefore, there remains a need for an antibacterial composition for application to skin that can be detected after use. There particularly remains a need for an antibacterial composition that children will accept and find entertaining in its application and detection.

### SUMMARY OF THE INVENTION

The present invention is an antibacterial composition that is safe for application to human skin and that contains a UV-detectable additive. The advantage of the UV-detectable additive is that it can be detected for some time following the application of said composition using a UV-light source, allowing for reapplication if complete coverage is not detected by UV.

### DETAILED DESCRIPTION OF THE INVENTION

There are many compounds that are antimicrobial and can be used in a liquid or colloid for application to surfaces. Those that are useful in the present invention are safe for application to human skin, act quickly to reduce the population of bacteria and other microbes after application, and are compatible with UV-detectable additives. In the following description of one type of sanitizer composition embodying the present invention, all percentages are express as percent by weight.

Among useful active ingredients in personal care products are quaternary ammonium compounds. These include benzalkonium chloride and benzethonium chloride. Most preferred is benzalkonium chloride. The effective amount for the active ingredient may be determined by one of ordinary skill in the art, but it is known that about 0.10 to about 0.13 percent is both safe and effective, with about 0.12 to about 0.13 percent being the most preferred amount of benzalkonium chloride. An example of a commercially available benzalkonium chloride product is Nobac® BZK NF from Pilot Chemical.

There are many different mixtures known to be useful as antibacterial compositions for personal care. It is generally preferred that they do not contain alcohol which tends to dry the skin upon application. Some examples of commercial products on the market today are Smart-San® Hand Sanitizer, Purell® Hand Sanitizer, and Germ-X® Hand Sanitizer. One of ordinary skill in the art can determine the compatibility with a UV-detectable additive to prepare a composition of the present invention.

There are many known UV-detectable compounds. Such compounds are preferably water soluble compounds that are fluorescent. For the present invention, this additive must be safe for application to the body. Examples of such additives are some of the colorants used in lipstick and other cosmetics. Such colorants can be found in the FDA's Color Additive Status List that may be found at https://www.fda.gov/forindustry/coloradditives/coloradditiveinventories/ucm106626.htm. Sources of the colorants useful in the present invention may be easily determined. Examples may be obtained from Day-Glo Colorant Corporation in Cleveland, Ohio. The products can be seen at www.dayglo.com, and UV-detectable additives can be selected from their product list of FDA-approved colorants. One of ordinary skill in the art can identify other sources for UV-detectable additives.

Examples of acceptable UV-detectable compounds are the colorants known commonly as D&C Green 8, D&C Red 22, and D&C Red 28. Green 8 is preferred for aesthetic reasons.

The effective amount of the UV-detectable additive in the present invention can be determined from routine experiments by one of ordinary skill in the art. Some additives may have upper limits set by the FDA for safe use on human skin. For example, FD&C Green 8 has an allowed upper limit of 0.1 percent of the final composition. A preferred amount is in the range of 0.003 to 0.010 percent, preferably between 0.005 and 0.008 percent, and most preferably around 0.006 percent.

Other inactive ingredients in the water-based embodiment of the present invention may include one or more surfactants, glycerine, polymers that act as coalescing agents, a preservative, and ingredients to adjust the pH of the final product. The water used is preferably deionized.

The surfactants can be any type to increase the viscosity and/or miscibility, but it is preferred that at least one produce a foaming effect. The amount of total surfactants used may be in the range of about 0.05 to about 5.0 percent, preferably in the range of 0.1 to 0.3 percent, and most preferably about 0.20 percent. Examples of acceptable surfactants include, but are not limited to Caltaine® C-35 from Stepan Chemical, Pluronic® L44 from BASF Corporation, Amphosol® CA from Stepan Chemical, Chembetaine™ C from Lubrizol, and the Macat® surfactants from Pilot Chemicals.

Examples of polymers that act as coalescing agent include, but are not limited to, Dowanol® ePh from Dow Chemical, phenoxyethanol from Galaxy Surfactants, Bronidox® 1160 from BASF, and Phenoxetol® from Clariant Ltd.

A broad spectrum preservative is an optional, but desirable, component in order to prevent growth of organisms that are not controlled by the antimicrobial active ingredient. An example of an acceptable preservative is Geogard® 221 from Lonza Ltd., which contains dehydroacetic acid, benzyl alcohol, and water. It may be used in an amount between about 0.1 to about 1.0 percent of the final composition. In an example of the product of the present invention, it is used at a level of 0.10 percent by weight.

The pH of the final composition should be in the range of 5.0 to 7.0. In one example of the product of the present invention, the pH is preferred to be around 5.5. Adjusting the pH is well within the skill of one of ordinary skill. It may be accomplished with, for example, high purity sodium hydroxide to lower the pH, or any acidic compound acceptable for application to human skin to lower the pH, for example, citric acid.

The advantage of the present invention is the ability to detect the occurrence and scope of application of antimicrobial compositions on the skin of humans. To do this, one uses a UV light source to detect the UV-detectable additive. Particularly desirable is a small light source that can be carried and used away from home in situations where hand sanitizer are most likely to be needed and used. Example of such a small UV light source can be readily found, for example, on the Amazon shopping website.

### EXAMPLE 1

The following is the formula of one example of an antibacterial composition that is safe for application to human skin and that contains a UV-detectable additive.

| Material | Percent by weight |
|---|---|
| Deionized water | 91.144 |
| Caltaine C-35 | 0.10 |
| USP Kosher Glycerine | 0.20 |
| Pluronic L-44 | 0.10 |
| Lonza Geogard 221 | 0.10 |
| Nobac BZK NF (50% benzalkonium chloride) | 0.25 |
| Dowanol ePh | 0.10 |
| D&C Green 8 | 0.006 |
| High purity sodium hydroxide or | As needed for pH |
| Citric acid food grade anhydrous | |

All patents and publications referenced herein are incorporated by reference. The invention has been described in an illustrative manner, and it is to be understood that the terminology which has been used is intended to be in the nature of words of description rather than limitation. Obviously, many modifications and variations of the present invention are possible in light of the above teachings. It is, therefore, to be understood that within the scope of the appended claims, the invention may be practiced otherwise than as specifically described above.

1-16. (canceled)

17. An aqueous solution for application to human skin as a skin sanitizer and protectant having a pH of between 5.0 to 7.0 and consisting essentially of:
zero percent by weight of the solution of alcohol, the zero percent alcohol not including trace amounts of benzyl alcohol,
an effective amount in the range of about 0.1 to about 0.13 percent by weight of the solution of an active microbial consisting of benzylkonium chloride,
0.003 to 0.020 percent by weight of the solution of a UV-detectable colorant,
0.1 to 0.3 percent by weight of the solution of a first surfactant and a second surfactant, the first surfactant having a foaming effect,
0.2 percent by weight of the solution of a first coalescing agent comprising a glycerine compound,
0.1 percent by weight of the solution a second coalescing agent comprising a glycerine compound, and
0.1 to 1.0 percent by weight of the solution of a broad spectrum preservative to prevent growth of organisms that are not controlled by the active microbial compound.

18. The aqueous solution of claim 17, wherein the UV-detectable colorant is D&C Green 8.

19. The aqueous solution of claim 18, wherein the UV-detectable colorant is between 0.005 to 0.008 percent by weight of the solution.

20. The aqueous solution of claim 19, wherein the UV-detectable colorant is 0.006 percent by weight of the solution.

21. The aqueous solution of claim 18, wherein the first surfactant is 0.1 percent by weight of the solution.

22. The aqueous solution of claim 21, wherein the second surfactant is 0.1 percent by weight of the solution.

23. The aqueous solution of claim 22, wherein the first surfactant is Caltaine C-35 and the second surfactant is Pluronic L-44.

24. The aqueous solution of claim 18, wherein the benzylkonium chloride is about 0.12 to about 0.13 percent by weight of the solution.

25. The aqueous solution of claim 24, wherein benzylkonium chloride is Nobac BZK NF 50 percent benzalkonium chloride.

26. The aqueous solution of claim 24, wherein the first coalescing agent comprising a glycerine compound is USP Kosher Glycerine and the second coalescing agent comprising a glycerine compound is Dowanol ePh.

27. The aqueous solution of claim 24, wherein the broad spectrum preservative is 0.1 percent by weight of the solution.

28. The aqueous solution of claim 27, wherein the broad spectrum preservative dehydroacetic acid and benzyl alcohol.

29. The aqueous solution of claim 27, wherein the aqueous solution has a pH of about 5.5.

30. An aqueous solution for application to human skin as a skin sanitizer and protectant having a pH of between 5.0 to 7.0 and consisting of:
zero percent by weight of the solution of alcohol, the zero percent alcohol not including trace amounts of benzyl alcohol,
an effective amount in the range of about 0.12 to about 0.13 percent by weight of the solution of an active microbial consisting of benzylkonium chloride,
0.006 percent by weight of the solution of a UV-detectable colorant,
0.1 percent by weight of the solution of a first surfactant, the first surfactant having a foaming effect,
0.1 percent by weight of the solution of a second surfactant,
0.2 percent by weight of the solution of a first coalescing agent comprising a glycerine compound,
0.1 percent by weight of the solution of a second coalescing agent comprising a glycerine compound, and
0.1 percent by weight of the solution of a broad spectrum preservative to prevent growth of organisms that are not controlled by the active microbial compound.

31. The aqueous solution of claim 30, wherein the UV-detectable colorant is D&C Green 8.

32. The aqueous solution of claim 31, wherein the first surfactant is Caltaine C-35 and the second surfactant is Pluronic L-44.

33. The aqueous solution of claim 32, wherein benzylkonium chloride is Nobac BZK NF 50 percent benzalkonium chloride.

34. The aqueous solution of claim 33, wherein the first coalescing agent comprising a glycerine compound is USP Kosher Glycerine and the second coalescing agent comprising a glycerine compound is Dowanol ePh.

35. The aqueous solution of claim 34, wherein the broad spectrum preservative comprises dehydroacetic acid and benzyl alcohol.

36. The aqueous solution of claim 30, wherein the aqueous solution has a pH of about 5.5.
